(19) United States
(12) Patent Application Publication
(10) Pub. No.: US 2010/0152844 A1
(43) Pub. Date: Jun. 17, 2010

(54) ANNULOPLASTY RING WITH DIRECTIONAL FLEXIBILITIES AND RIGIDITIES TO ASSIST THE MITRAL ANNULUS DYNAMICS

(76) Inventor: Jean-Paul A. COUETIL, Abu Dhabi (AE)

Correspondence Address: OLIFF & BERRIDGE, PLC, P.O. BOX 320850, ALEXANDRIA, VA, 22320-4850, US

(21) Appl. No.: 12/335,177

(22) Filed: Dec. 15, 2008

(51) Int. Cl. A61F 2/24

(52) U.S. Cl. 623/2.36

(57) ABSTRACT

A prosthetic ring with directional flexibilities and rigidities for use in minimally invasive or standard mitral valve repair includes an anterior segment, a posterior segment, a left lateral segment, and a right lateral segment. The anterior and posterior segments each have an arch, and the arch of the posterior segment is more pronounced than the arch of the anterior segment. The prosthetic ring is formed in a continuous D shape, with an inner body that generally comprises a flexible or semi-flexible material, such as polyethylene or nitinol or other biocompatible material. The inner core, when made of a plastic material, may be reinforced by arched strips of nitinol in the anterior and posterior segments. The lateral segments are thin and fully flexible to allow passive deformation that follows the normal physiological dynamics of the native annulus during the cardiac cycle.

### FIELD OF INVENTION

The present invention relates to prosthetic annuloplasty rings for a mitral valve for use in heart surgery and more particularly annuloplasty rings for use in minimally invasive or standard mitral valve repair, and methods for using the annuloplasty rings.

### BACKGROUND

The left atrioventricular heart valve, or mitral valve, is located between the left atrium and left ventricle. During normal physiological function, the mitral valve allows oxygenated blood to pass from the atrium to the ventricle. During systole, the mitral valve closes, and prevents blood from regurgitating into the left atrium. The mitral valve may become damaged by aging or by pathologies such as rheumatic disease, endocarditis, fibroblastic or myxomatous degeneration, Barlow's disease, etc. As a result of such damage, the mitral valve no longer functions properly and efficiently.

The mitral valve may be structurally damaged during or after certain diseases, or may exhibit regurgitation that is characterized by backflow of blood from the ventricle to the atrium. When the mitral valve is no longer functioning normally, the valve may be replaced by a prosthesis or other replacement material, or more and more commonly, repaired by surgical or percutaneous mitral annuloplasty.

Mitral valve repair has been performed for over 40 years and has numerous advantages over valve replacement. It may combine annuloplasty and specific surgical procedures directed to the elements of which the mitral valve is composed, e.g., the mitral leaflets, chordae tendinae and papillary muscles.

A number of mitral prostheses have been described in U.S. Patent Application Publications Nos. 2008/0086203, 2008/0058924, and 2006/0184240, and in U.S. Pat. Nos. 6,602,289, 6,217,610, 6,187,040, 5,716,397, 5,607,471, 5,593,424, 5,376,112, 5,306,296, 5,163,954, 5,104,407, 5,064,431, 5,061,277, 4,917,698, 4,489,446, 4,290,151, 4,164,046, 4,042,979, and 3,656,185. The rings of these mitral prostheses are rigid or fully or partially flexible, planar or three-dimensional, and closed or open. Other than the fully flexible closed or partial band prosthetic rings, the flexibility of other rings has never been demonstrated in vivo. Mitral prosthetic rings are used to set and maintain the shape and circumference of the native mitral annulus and to restore good coaptation of the valve leaflets. Recent rings are formed with a saddle-shape to restore geometry of the native mitral annulus that is more physiological (see Levine, Circulation 1999; 80:589-598). These prosthetic rings are usually secured to the native mitral annulus by threads passing through the native ring and engaging a peripheral DACRON® (polyester textile) sleeve or other polyester sheath enveloping the prosthetic ring structure.

Experimental and clinical studies over the last ten years have refined the knowledge of the shape and dynamics of the native mitral annulus in humans and animals under physiological and pathological conditions during the cardiac cycle. After closure of the mitral valve, the valve leaflets and mitral annulus have a saddle shape, and the annulus itself has a substantially hyperbolic parabolic shape with an upwardly arched appearance and anterior and posterior segments that define the height of the annulus. This height is generally greater for the anterior segment than for the posterior segment (see Timek, Circulation 2005, 112 (Suppl): I-423-I-428) and varies by about 15 to 20% in the course of the cardiac cycle. The annulus is flatter during diastole than during systole and will flatten if there is mitral insufficiency The septolateral and intercommissural diameters of the native annulus also vary during the cardiac cycle.

It has also been demonstrated that most known prosthetic rings have a negative effect on the functioning of the mitral apparatus (e.g., restriction in mobility of the posterior valve and changes in forces applied to the chordae).

### SUMMARY

Embodiments of the present invention comprise a non-planar prosthetic ring with at least one segment having directional rigidity and flexibility, at least one rigid or semi-rigid segment, and at least two pliable, supple, and fully flexible segments. The prosthetic ring may be continuous or discontinuous. For example, the prosthetic ring may be a continuous ring, in which four segments are integrally (e.g., unitarily) joined at their ends at a plurality of junctions. Alternatively, for example, the prosthetic ring may be a discontinuous ring, in which at least one end of a segment is not integral with an end of another segment.

In embodiments, because of the fully flexible and pliable mechanical nature of the lateral segments, at least one of which may be not integral with the anterior segment, the prosthetic ring has passive systolodiastolic deformation properties that offer little or no resistance to movement, and respects and replicates the active systolodiastolic deformation amplitude of the native mitral annulus at the commissural zones and adjacent portions.

Computer modeling and simulation studies (e.g., computational modeling) have shown that the amplitude of the deformations of the lateral segments may be 4 to 5 mm when subject to forces less than or equal to 1 Newton, and may be larger when subject to forces greater than 1 Newton.

### BRIEF DESCRIPTION OF THE DRAWINGS

FIG. 1 is a schematic representation of an example of a prosthetic mitral ring in projection in the horizontal plane XoY with its four segments, an arcuate anterior segment, an arcuate posterior segment, and two lateral segments;

FIG. 2 is a schematic representation of a prosthetic ring in projection in the sagittal plane YoZ;

FIG. 3 is a schematic representation of a prosthetic ring in projection in the frontal plane XoZ;

FIG. 4 is a schematic representation of a prosthetic ring with four segments, depicted in three dimensions and illustrating a discontinuity in the prosthetic ring;

FIGS. 5A and 5B are schematic representations of the projections of a prosthetic ring in a sagittal projection that illustrate the height of the anterior arch h_{A}, the maximum anterior height H_{A} of the prosthetic ring, the height of the posterior arch h_{P}, and the maximum posterior height of the prosthetic ring H_{P};

FIG. 6 is a schematic representation of a prosthetic ring, depicted in three dimensions, and illustrates the downward displacement of the right lateral segment toward the tip of the left ventricle, under the action of forces developed during systole;

FIGS. 7-7E are schematic representations of a prosthetic ring, depicted in three dimensions, and illustrates the downward displacement of the right lateral segment toward the tip of the left ventricle, and of the left lateral segment under the action of the forces developed during systole, and that illustrate cross sections of the prosthetic ring;

FIG. 8 is a schematic representation of an example of a prosthetic mitral ring in projection in the horizontal plane XoY with four segments: an arcuate anterior segment with two lateral slits, an arcuate posterior segment, and two lateral segments;

FIG. 9 is a schematic representation of a prosthetic ring in projection in the sagittal plane YoZ with a slit disposed in the lateral part of the anterior segment;

FIG. 10 is a schematic representation of a prosthetic ring in projection in the frontal plane XoZ with two lateral slits arranged in the lateral parts of the anterior segment;

FIGS. 11-11E are schematic representations of a complete continuous prosthetic ring with an anterior segment having slits disposed in its lateral parts, depicted in three dimensions, and illustrate various types of slits disposed in the lateral parts of the anterior segment of the prosthetic ring;

FIG. 12 is a schematic representation of an asymmetric prosthetic ring having lateral segments of unequal length; and

FIGS. 13A and 13B are schematic representations of the projections of a prosthetic ring in a sagittal projection that illustrate the height of the anterior arch h_{A}, the maximum anterior height H_{A} of the prosthetic ring, the height of the posterior arch h_{P}, and the maximum posterior height of the prosthetic ring Hp when the prosthetic ring is subjected to downward forces that open slits disposed in the lateral parts of the anterior segments.

### DETAILED DESCRIPTION OF EMBODIMENTS

Embodiments include a complete, preferably continuous three-dimensional prosthetic ring for a mitral annuloplasty that may be used in standard or minimally invasive heart surgeries and is intended to be secured to the native mitral annulus. The prosthetic annuloplasty ring has a three-dimensional shape with four segments: an anterior segment, a posterior segment, a left lateral segment, and a right lateral segment. The anterior and posterior segments each have an arch. The arch of the anterior segment is preferably more pronounced than the arch of the posterior segment. These segments have an upper face, a lower face, an inside face and an outside face for the anterior and posterior segments, and an upper face, a lower face, an inside edge and an outside edge for the lateral segments.

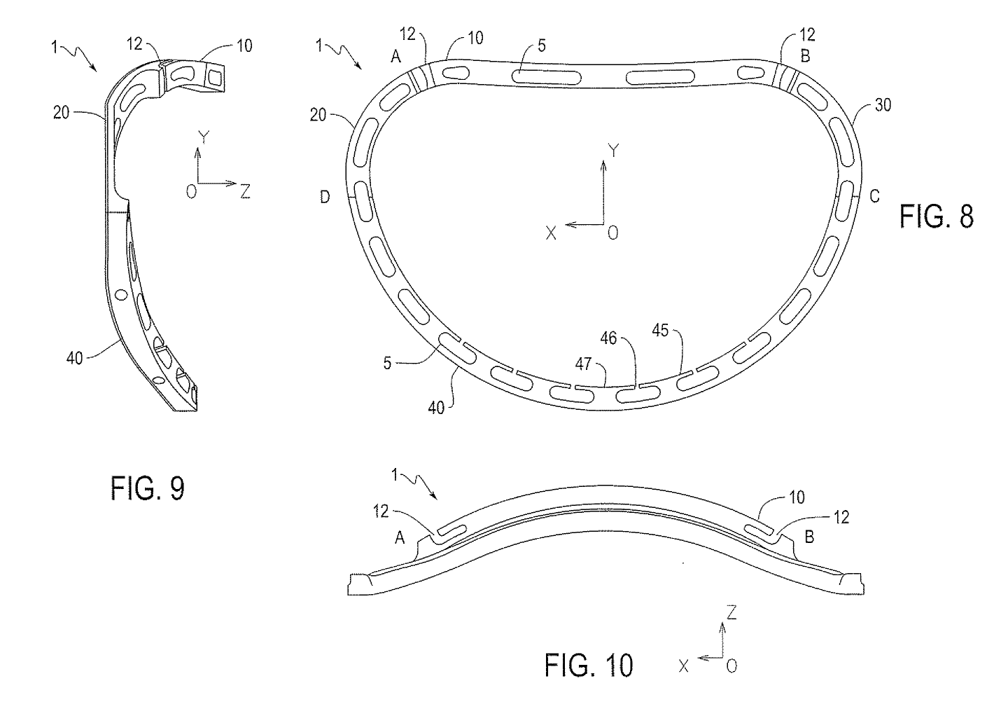

The prosthetic ring is preferably formed with a continuous D shape, with an inner body that preferably comprises a biocompatible material with elastic properties, for example a metallic alloy such as nitinol or elgiloy or a polymer such as polyethylene, polyurethane or other biomaterial with flexible properties. When made of biocompatible plastic material, the inner core may be reinforced by thin arched strips of a metal such as nitinol. For example, such strips may be placed in the anterior and posterior segments, respectively, to maintain the respective arch. The configuration of the strips allows the arches to be rigid in one plane and resistive to distortion when subjected to a stress applied in that plane, but flexible or semi flexible in a perpendicular plane. Conversely, the lateral segments are thin and intrinsically fully flexible in at least one plane, to allow adequate passive deformation that follows the normal physiological dynamics of the native annulus during the cardiac cycle. This allows the height of the ring to increase during systole and conform to the normal physiological dynamics. The anterior and posterior arches conform to the diastolic mitral annulus shape to decrease the stress on sutures that may be used for anchoring the ring to the annulus and to reduce the risk of dehiscence. The inner core of the anterior segment preferably has one or more slits located close to ends A and B of the anterior segment, that create two sub-segments, which allow the lateral parts of the anterior segment to have a desirable flexibility in one direction and a desirable rigidity in the opposite direction (FIG. 8). The inner core of the posterior segment preferably has multiple slits that create pseudo-segments that allow the inner core to have a desirable flexibility in one direction and a desirable rigidity in another direction. The ring generally has a very low profile structure to minimize the fibrotic reaction that may impair its mechanical properties after implantation, and may have multiple holes through which sutures may pass for anchoring the ring in the mitral annulus.

The body of the ring is configured to be implanted and arranged around an axis of blood flow, to allow the blood to flow in the downward direction, from the left atrium to the left ventricle.

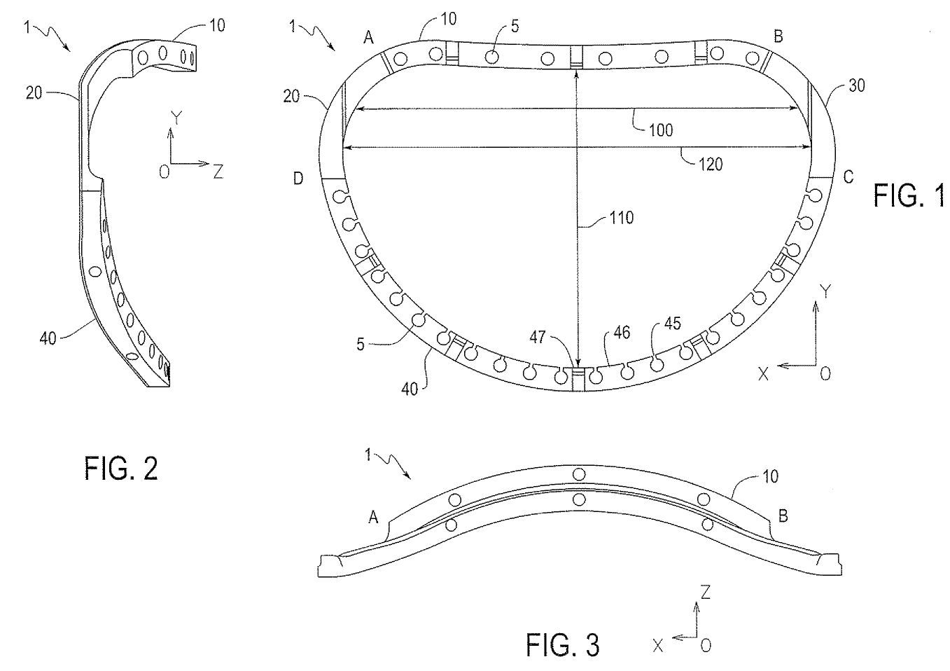

The prosthetic ring projects in three orthogonal planes: XoY, YoZ and XoZ. The XoY plane, referred to as the “horizontal” plane, is substantially perpendicular to the blood flow axis, and is arbitrarily defined as being a plane where the projections of the intercommissural diameter, the septolateral diameter, and the major horizontal diameter of the ring are the greatest. The projection of the prosthetic ring in the XoY plane has the general shape of a capital “D” (FIG. 1). Three dimensions or distances of the ring are defined in the XoY plane (i.e., the intercommissural diameter 100, the septolateral diameter 110 and the major transverse horizontal diameter 120). The intercommissural diameter 100 passes through the projection of the lowest points on the prosthetic ring and cuts perpendicularly the septolateral diameter 110 in two equal portions between the projection of the anterior segment 10 of the prosthetic ring and the major transverse horizontal diameter 120. The septolateral diameter 110 (minor axis) passes through the projection of the highest points on the ring. The major horizontal diameter 120 (major axis) passes through the farthest lateral points on the ring, is perpendicular to the septolateral diameter 110, and is posterior to the intercommissural diameter 100 (FIG. 1).

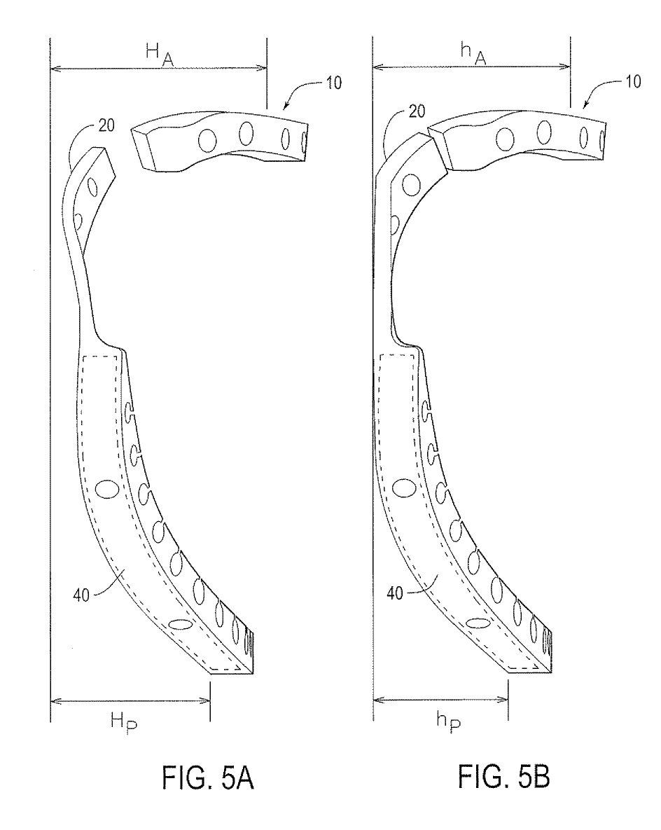

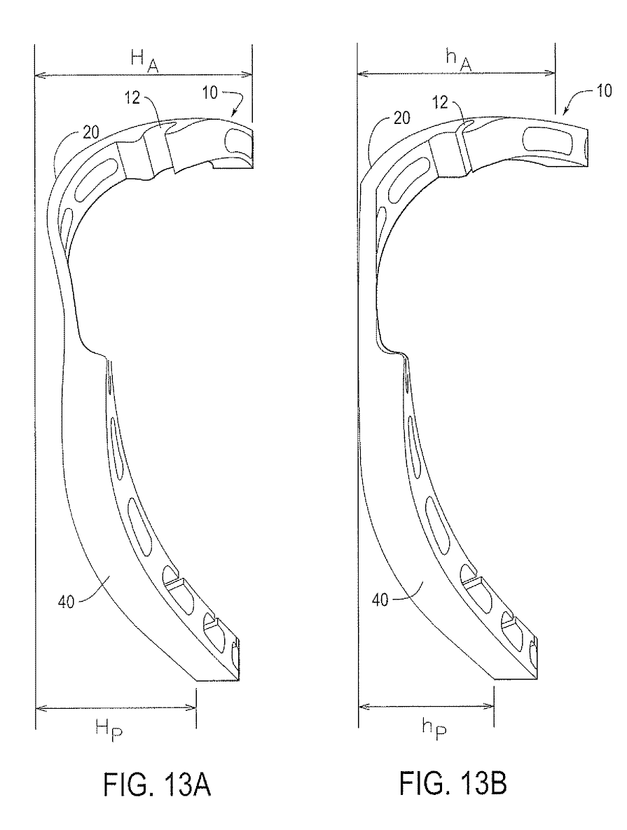

The YoZ plane, referred to as the “sagittal” plane, is perpendicular to the XoY plane, passes through the highest points on the anterior and posterior segments and intercepts these anterior and posterior segments at their centers (FIG. 2). The sagittal plane defines the anterior height of the ring H_{A} greater than or equal to the height of the arch of the anterior segment h_{A}, as well as the posterior height of the prosthetic ring H_{P} greater than or equal to the height of the arch h_{P} of the posterior segment (FIGS. 5A, 5B, 13A, 13B). The height of the arch of the anterior segment h_{A} also is referred to as the major height, and the height of the posterior segment hₚ also is referred to as the minor height (FIGS. 5B and 13B). In embodiments in which the prosthetic ring is continuous and the lateral segments are of equal length, the sagittal plane may cut the ring into two identical symmetrical parts, the left and right parts. In embodiments in which the prosthetic ring is continuous and the lateral segments are of unequal length, the sagittal plane may divide the ring into two asymmetrical parts, the left and the right parts.

The XoZ plane, referred to as the “frontal” plane, is perpendicular to both the horizontal and sagittal planes, and is the plane on which the anterior and posterior arched segments are projected in their major axes (FIG. 3).

In embodiments, the prosthetic ring has a body made of a flexible or semi-rigid biocompatible material of the polyethylene or polyurethane type or metallic alloy type, having an upwardly arched anterior segment rigid along the frontal plane and semi-rigid or rigid in the direction of the horizontal plane, and having two ends, A and B. The upwardly arched anterior segment may be provided with at least one slit close to each of the ends A and B, creating an opposing directional flexibility and rigidity along the frontal plane between two sub-segments that cooperate by means of a linking element at elastic inferior hinge zones. When a force directed downward (toward the Z direction) is applied to the ring, the slits may open, making the anterior segment flexible in this direction. The lateral parts of the anterior segment and consequently the lateral segments, at least in their portions that are close to the ends of the anterior segment, may then move downward and inward, increasing the height of the anterior segment and thereby reducing the intercommissural distance. This represents one of the physiological deformations of the native mitral annulus during the cardiac cycle. When the above mentioned forces are no longer applied toward the apex of the left ventricle, the ring passively returns to its initial resting position due to its elastic properties. The ring cannot move beyond its resting position in the superior direction because the slits bring the sub-segments into contact with each other as they close, thereby stiffening the body of the anterior segment and preventing its upward excursion. The rigidity of the ring prevents any shortening of the height of the anterior arch, and hence, any segmentary flattening of the native mitral annulus.

In embodiments, ends A and B of the anterior segment are preferably continuous with the adjacent lateral segments. In embodiments, end B of the anterior segment may have a discontinuity with its adjacent lateral segment. The supple, pliable and intrinsically flexible lateral segments extend to the arched posterior segment. The lateral segments have rigidity along the horizontal plane and full flexibility along the sagittal plane When physiological systolic forces are applied to the lateral segments, the lateral segments flex about a horizontal axis that passes through the commissural zones in the horizontal plane. The lateral segments bow downwardly and move towards the apex of the left ventricle in the downward Z direction of the sagittal plane and thereby the height H_{A} of the ring may be lengthened and the anterolateral diameter 110 may be reduced. The posterior segment can deform in the direction of the horizontal plane under the influence of a centripetal force that is directed toward the inside of the ring, and thereby the septolateral diameter 110 may be reduced when ventricular systole occurs.

The upwardly arched posterior segment may be provided with slits or crenellations on its inside face, creating pseudo-segments between them, which cooperate by means of linking elements at elastic posterior hinge zones.

When a centripetal force directed toward the inside of the ring is applied to this ring, the slits may open, making the posterior segment flexible in this direction. The posterior segment then may move toward the inside of the ring, reducing the septolateral distance 110. This represents one of the physiological deformations of the native mitral annulus during the cardiac cycle. When the centripetal force is no longer applied toward the inside of the ring, the ring passively returns to its initial resting position due to its elastic properties. The ring cannot move beyond its resting position in the outward direction because the slits bring the pseudo-segments into contact as they close, stiffening the body of the posterior segment and blocking its outward excursion. The rigidity of the ring prevents any elongation along the septolateral axis, and hence, any segmentary dilation of the native mitral annulus.

In embodiments, the core of the ring may be made of a biocompatible elastic polymer, and the anterior and posterior segments may each have a very thin arcuate metal reinforcement made of nitinol, or other elastic metal or alloy, included in the body of the ring. The strips may be cut to their desired length before being placed within the ring and prior to the ring being polymerized. In embodiments, when the strips of the ring are made of nitinol, the thickness of the strips is preferably about 0.1 mm and their height is nearly the thickness of the body of the ring. The configurations of the reinforcements included in the body of the ring are such that the anterior and posterior segments remain non-deformable and rigid in the direction of the frontal plane, while the small thickness and elasticity of the reinforcements do not alter the deformability and mechanical properties of the anterior and posterior segments in the horizontal plane. The reinforcements maintain and fix the respective heights of the two arcuate segments. Whatever the material of which the core of the ring is made, in the original resting configuration of the unstressed prosthetic ring, the relative heights of the anterior and posterior arches preferably represent 10% to 25% of the intercommissural diameter 100 of the ring. This original shape, known as the resting shape, corresponds to the morphology of the native mitral annulus during diastole. The height of the prosthetic ring varies during the cardiac cycle to passively adapt to the height variations of the native mitral annulus, due to the very high flexibility and pliable nature of the lateral segments and the directional flexibility of the lateral parts of the anterior segment. Thus, the heights H_{A} and H_{P} of the prosthetic ring in systole become greater than the heights of the anterior and posterior arcuate segments h_{A} and h_{P} and increase from 0 to 6 mm as a function of the forces applied to these lateral segments, as shown in modeling studies of a prosthetic ring when subjected to physiological forces that are specifically applied to the lateral segments. The relative heights of the prosthetic ring in systole represent 25% to 40% of the length of the intercommissural diameter 100 of the ring. The discontinuity between the anterior segment and the right lateral segment when present, increases the flexibility of the latter segment and respects the mobility of the anterior commissure of the native mitral annulus.

A plurality of holes may pass through the lower and upper faces of the ring body. These holes, which are preferably but not necessarily evenly distributed, allow for suturing the prosthetic ring to the native mitral annulus. Particularly with a plurality of holes that are numerous and closely spaced, it may not be necessary to flag the holes by marks on a sleeve that may envelop the body of the ring. The holes provide excellent anchoring for the prosthetic ring to the native mitral annulus and thus eliminate the need for an additional silicone layer, which is used with conventional rings between the outer envelope and the body of the ring. The small dimensions of the cross section of the prosthetic ring body contribute to its very low profile, and thus make the size of the prosthetic ring suitable for children and effective to reduce the fibrosis reaction that may surround the ring after implantation. In a preferred embodiment, the cross section dimensions of the anterior and posterior segments may be around 1.2 mm in thickness and 1.6 mm in width, particularly when the core is made of an elastic metallic alloy. The thickness of the lateral segments may be minimal, for example at least 0.1 mm. The above discussed dimensions are only exemplary, as the dimensions may vary substantially, provided that the suitable mechanical properties of the different segments of the core are maintained.

During conventional mitral annuloplasty procedures, many surgeons implant a prosthetic ring whose size corresponds to the systolic dimensions of the native mitral annulus. To determine the appropriate size of the prosthetic ring, surgeons conventionally place a prosthetic annulus-measuring device over the anterior mitral leaflet to obtain the systolic dimensions of the native mitral annulus. Drawbacks to this conventional method may include (1) placing a ring that is too small relative to the actual dimensions of the native annulus, which may result in as much as a 50% reduction of the native mitral annulus surface area; (2) favoring dehiscence at the suture stitches and anchoring points of the prosthetic ring; (3) creating a slight transmittal gradient and impeding the filling of the left ventricle; (4) restricting or blocking movements of the posterior mitral valve when the quantity of posterior valve tissue is normal or insufficient after partial resection; and (5) displacing the coaptation zone of the valve leaflets in a direction toward the ejection chamber of the left ventricle and thus creating systolic anterior motion when there is excess valve tissue.

This conventional method superimposes a prosthetic-measuring ring device (PMRD), which is manufactured, as well as the prosthetic ring, with a ratio of 3:4 between the septolateral diameter 110 and the major transverse horizontal diameter 120 of the ring. The PMRD is superimposed over the anterior mitral valve leaflet and has a ratio between its height and its largest diameter (intercommissural diameter) that is greater than 3:4. As a consequence, the selected ring is undersized. The undersizing of the selected ring is a result of the facts that: 1—the PMRD covers the anterior mitral leaflet whose surface area is less than the systolic surface area of the native mitral valve, and 2—by matching the major transverse horizontal diameter of the PMRD with the intercommissural diameter of the native annulus, the PMRD partially covers the anterior mitral leaflet. These factors in turn influence a surgeon to select a ring that is undersized for the desired treatment.

In contrast with the known mitral valve repair techniques described above, embodiments of prosthetic rings and methods according to the present invention involve implanting and securing the ring to the native mitral annulus in the diastolic position, and not in the systolic position. Thereby, an optimal diastolic mitral surface for diastolic filling of the left ventricle is determined, consequently eliminating the pressure gradient generated by the conventional technique of systolic superimposition of the prosthetic ring over the native mitral annulus.

Embodiments of prosthetic rings and methods of the present invention provide an increased physiological ratio between the septolateral and the intercommissural diameters 110 and 100 that is at least greater than a conventional ratio of 3:4 (calculated from the septolateral diameter 110 and the major transverse horizontal diameter 120, and preferably at least equal to a conventionally calculated ratio of 3.2:4 for any mitral insufficiency whose relative or absolute height of the anterior and posterior valve leaflets is at least equal to the length of the insertion of the anterior mitral leaflet on the native annulus, and not just for certain pathologies such as Barlow's disease which is known to involve excess valve tissue, as described in U.S. Patent Application Publication No. 2008/0058924. The conventional rings are manufactured with reference to a ratio of two diameters: the anterolateral diameter and the major transverse horizontal diameter of the conventional rings. Moreover, in the conventional surgical approach to determine the appropriate size of the prosthetic ring to be implanted, surgeons place a prosthetic annulus-measuring device over the anterior mitral leaflet to obtain the systolic dimensions of the native mitral annulus. With this approach a surgeon may measure the major diameter of the anterior leaflet of the mitral which corresponds exactly to the intercommissural diameter of the native mitral annulus which, as already stated, is shorter than the major transverse horizontal diameter of the native mitral annulus.

In the conventional method, there is confusion among 1—the intercommissural diameter of the native mitral annulus, 2—the major transverse horizontal diameter of the native mitral annulus on the basis of which the manufacturing of the rings is established, and 3—the major diameter of the anterior leaflet which matches the intercommissural diameter of the native mitral annulus on the basis of which the size of the implanted ring is chosen. As a consequence, the chosen mitral ring is undersized and does not match the native mitral annulus resulting in the above drawbacks. The conventional rings manufactured with a ratio of 3:4 between their major transverse diameter and their septolateral diameter are inadequately sized with the method of superimposing the measuring device over the anterior mitral leaflet, which is equivalent to downsizing the selected ring to a ratio inferior to 3:4, which in turn considerably reduces the mitral annulus surface area by up to 50%, as has been demonstrated in 3D echocardiography measurements.

In order to eliminate drawbacks related to conventional methods, embodiments of the present invention restore the shape and the 3D dimension of the native annulus, restore or maintain its physiologic motion, and restore a normal surface of coaptation and a normal mitral annulus surface area. Thus embodiments of rings of the present invention are manufactured with a ratio between their intercommissural diameter 100 and their septolateral diameter 110 that is in accordance with accurate reference diameters used to measure the true size of the native mitral annulus. The accurate reference diameters refer to the measured intercommissural diameter of the native mitral annulus and the measured heights of the leaflets of the mitral valve. The intercommissural diameter of the native annulus can be accurately measured on the pathological mitral valve and is a parameter that is not affected by diseases of the mitral valve, as it corresponds to the length of the anterior mitral leaflet insertion to the native annulus. Conversely, the septolateral diameter of the native mitral annulus may be modified and/or damaged by the pathological presentation of the mitral valve, and thus its direct measurement is not reliable because the mitral valve is usually dilated. In embodiments of the present invention, the assessment of the septolateral diameter of the native mitral annulus is based on accurate measurements of the height of the middle scallop of the posterior leaflet if it has not been resected, or is based on the height of an adjacent portion if resection has been carried out, and the height of the anterior leaflet in its median portion. To insure a good surface of coaptation of the two leaflets, the calculated dimension of the septolateral diameter of the native mitral annulus is about 80% to 90% of the sum of the measured heights of the anterior and posterior leaflets. As a result the length of this calculated septolateral diameter will be equal, less than, or greater than, the intercommissural diameter of the native mitral annulus. Thus the calculated septolateral diameter and the measured intercommissural diameter of the native mitral annulus are two accurate diameters that may be used to choose the ring that best matches the mitral annulus surface area.

Thus, in embodiments, rings of the present invention are manufactured according to a ratio between the septolateral diameter of the prosthetic ring 110 and the intercommissural diameter of the prosthetic ring 100, in contrast to the conventional rings that are manufactured according to a ratio between the septolateral diameter of the conventional ring and the major transverse horizontal diameter of the conventional ring. Because the heights of the mitral leaflets may vary from one patient to another, according to the underlying pathology, each ring of the present invention with one given intercoinmissural diameter 100 may be manufactured with one of three different septolateral diameters 110, such that the ratios between the septolateral diameter 110 and the intercommissural diameter 100 are less than 1, equal to 1, or greater than 1. The three different septolateral diameters that correspond to one given intercommissural diameter may provide the surgeon with flexibility in choosing a ring size to accommodate various physiological/pathologies conditions of the native mitral annulus/heart. For example, the ratio between the septolateral diameter 110 and the intercommissural diameter may be between 0.85:1 and 1:1, or the ratio may be between 1:1 and 1.15:1. The rings may be manufactured in various sizes, for example, the size of the ring may be in the range of 22 to 46 mm, which corresponds to the size of its intercommissural diameter 100. Further, the sizes of the rings may differ in various increments, for example, increments of 2 mm. For example, ring 30 may have a ratio between its septolateral diameter 110 and its intercommissural diameter 100 that is less than 1, equal to 1, or greater than 1.

While three-dimensional prosthetic rings that are either rigid or semi-rigid distort and constrain the native mitral annulus by keeping it in systolic position, prosthetic rings of the present invention, due to their excellent properties of passive deformation, that are variable at various segments, follow and conform to the systolodiastolic deformation of the native mitral annulus during the cardiac cycle. Rings of the present invention restore and maintain the physiological three-dimensional character as well as the systolodiastolic dynamic morphological variations of the native mitral annulus. Because of such mechanical properties, rings of the present invention also restore normal constraints on the valve tissue, chordae, and papillary muscles. Moreover, the directional deformability from the resting position of the posterior segment, in the horizontal plane and in the centripetal direction (toward the inside of the ring), allows physiological shortening of the septolateral axis 110 of the prosthetic ring during systole. The centrifugal directional rigidity (directed toward the outside of the ring) of the posterior segment in the horizontal plane from its resting position prevents any pathological dilation of the minor axis of the native mitral valve, and ensures the durability of the mitral valve repair quality. In addition, the directional deformability from the resting position of the lateral parts of the anterior segments, in the frontal plane and in the downward and inward directions, allows physiological shortening of the intercommissural diameter 100 as well as the lengthening of the heights of the prosthetic ring during systole. The upwardly directional rigidity of the anterior segment in the frontal plane from its resting position prevents any pathological flattening of the native mitral valve, and ensures the durability of the mitral valve repair quality.

In contrast with known prosthetic rings described above, and particularly in contrast with the ring described in the U.S. Patent Application Publication No. 2005/0256569, prosthetic rings and methods of the present invention have, in a given plane and in a given direction, true flexibility while exhibiting total rigidity in the same plane and in the opposite direction. This property allows restoration of the normal size and morphology, and restoration of the normal physiological dynamics, of the native mitral annulus.

In order to illustrate the complex shape and orientation of the prosthetic rings of the present invention, three orthogonal planes have been defined.

The XoY plane, called the “horizontal plane,” which is substantially perpendicular to the transmittal blood flow, is arbitrarily defined as the plane where the projections of the intercommissural diameter 100 and septolateral diameter 100 of the ring, are the greatest. The horizontal plane virtually separates the left ventricle from the left atrium, defines a downward direction on the ventricle side, and defines an upward direction on the atrium side, as shown in FIG. 1. Thus, an upwardly directed arch is on the atrium side.

The projection of the prosthetic ring in the XoY plane has the general shape of a capital “D.” The intercommissural and septolateral diameters 100 and 110 of the ring, or distances, may be defined in the XoY plane, whereby the intercommissural diameter 100 passes through the projections of the lowest points of the prosthetic ring and cuts perpendicularly the septolateral diameter 11 0 in two equal portions, such that the portion of the septolateral diameter 110 between the projection of the anterior segment 10 of the prosthetic ring in the plane XoY and the intercommissural diameter 100 is equal to the portion of the septolateral diameter between the intercommissural diameter 100 and the major transverse horizontal diameter 120. The septolateral diameter 110 or minor axis passes through the projections of the highest points of the ring. The major transverse horizontal diameter 120 (major axis) passes through the farthest lateral points on the ring, is perpendicular to the septolateral diameter 110, and is posterior to the intercommissural diameter 100.

The YoZ plane, called the “sagittal plane,” which is perpendicular to the XoY plane, passes through the highest points on the anterior and posterior segments and intercepts the anterior and posterior segments at their centers, as shown in FIG. 2.

The XoZ plane, called the “frontal plane,” which is perpendicular to the horizontal and sagittal planes, is the plane in which the anterior and posterior arched segments are projected in their major axis.

In embodiments, the prosthetic ring is closed and represents a complex three-dimensional geometric shape whose projection on the horizontal plane is defined in the shape of a capital letter “D.” The prosthetic ring may include four segments: a rigid or semi-rigid upwardly-arched anterior segment 10 in the frontal plane is configured to be secured to the native mitral annulus between the anatomical trigones, has two ends, and is extended by two flattened, flexible lateral segments 20 and 30, as shown in FIG. 3. This configuration allows greater deformability, while not being extendable, and allows the prosthesis to be secured to the commissures and to the adjacent zones. These symmetrical lateral segments 20 and 30 each have two ends, and are extended by the posterior segment 40, which is configured to be secured to the posterior native mitral annulus.

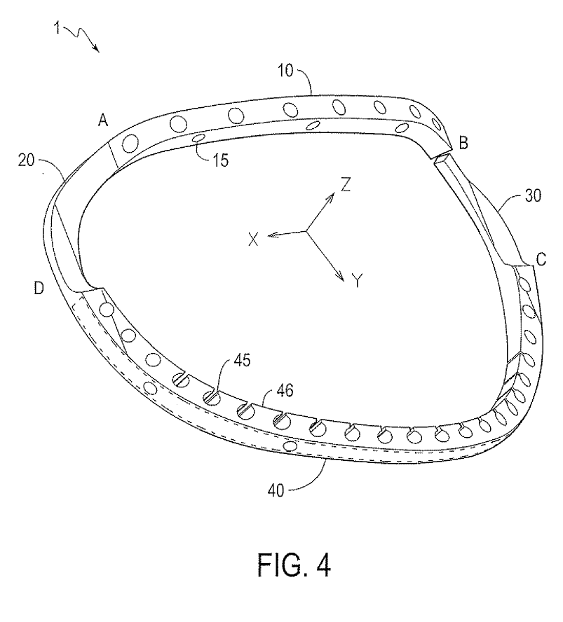

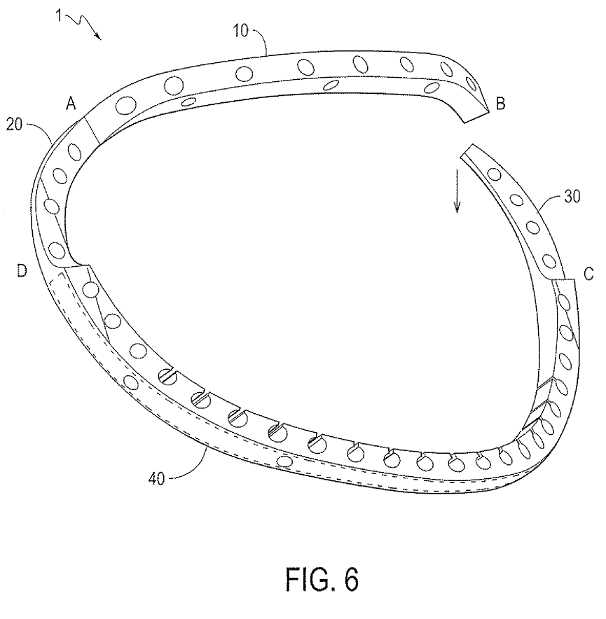

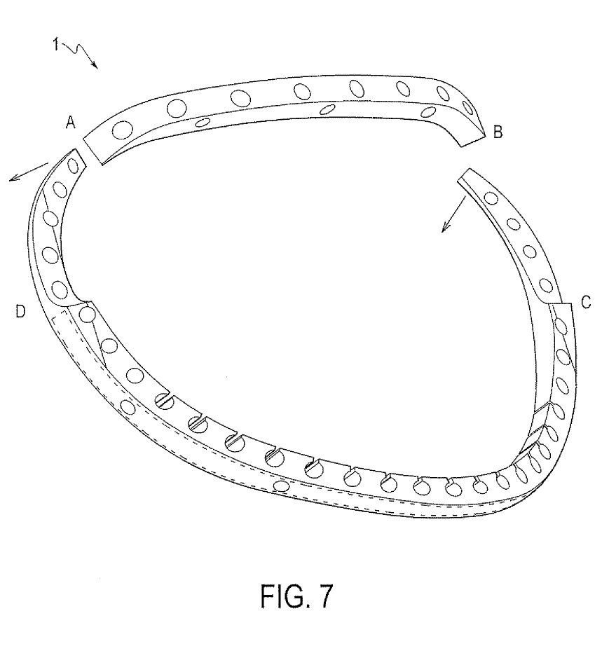

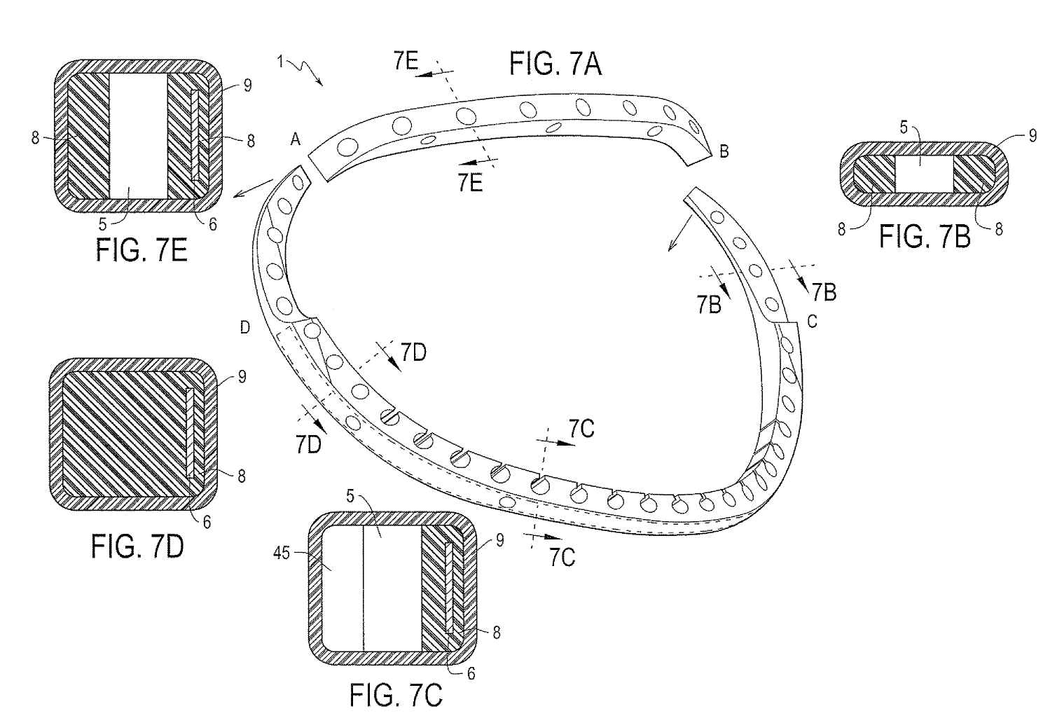

The prosthetic ring 1 may include four junctions A, B, C and D, as shown in FIGS. 1, 3, 4, 6 and 7. The first junction A is between a first end of the anterior segment 10 and a first end of the left lateral segment 20. The second junction B is between a second end of the anterior segment 10 and a first end of the right lateral segment 30. The third junction C is between a first end of the posterior segment 40 a second end of the left lateral segment 20. The fourth junction is between a second end of the posterior segment 40 and a second end of the right lateral segment 30. FIG. 4 is a schematic representation of an example of the present invention including a prosthetic ring with four segments, depicted in three dimensions and illustrating a discontinuity in the prosthetic ring. The prosthetic ring 1, shown in FIG. 1, is a continuous ring, in which the segments of the ring are joined by four junctions. The prosthetic rings 1 shown in FIGS. 4 and 6 are discontinuous rings, in which the four segments of the rings are joined by only three junctions. The prosthetic ring 1, shown in FIG. 7, is a discontinuous ring, in which three segments of the ring are joined by two junctions. The examples shown in FIGS. 4, 6 and 7 are exemplary, and the discontinuous rings of these figures may include various combinations of the segments being joined by the various junctions.

The posterior segment 40 is preferably arched upward in projection on the frontal plane, has two ends C and D, and has directional flexibility directed toward the center of the prosthetic ring 1, and has directional rigidity opposing any force directed toward the outside of the ring 1.

The posterior segment 40 may be provided with slits 45 that create between them pseudo-segments or crenellations 46 that cooperate through linking elements 47 and are articulated to each other at elastic posterior hinge zones of the ring body. When a centripetal force is applied toward the center of the ring 1, the slits 45 open, rendering the posterior segment 40 flexible in a direction toward the center of the ring 1. The posterior segment 40 then moves toward the center of the prosthetic ring 1, which reduces its septolateral dimension, and has been demonstrated in modeling studies of the prosthetic ring 1. This represents one of the physiological deformations of the native mitral annulus during the cardiac cycle. When the centripetal force disappears, the prosthetic ring 1 returns passively to its initial position and resumes its resting shape because of its elastic properties.

When a centrifugal force is applied to the posterior segment 40, the slits 45 close and bring the pseudo-segments 46 into contact, which stiffens the body of the posterior segment 40 and blocks its outward excursion. This rigidity resists any prolongation of the septolateral axis and hence any segmentary dilation of the native mitral annulus.

Slits 45 can be distributed regularly or irregularly along the posterior segment 40, over all or part of posterior segment 40. The depth of the slits 45 may be uniform or non-uniform.

In embodiments the anterior segment 1O has slits 15 disposed on its inside face or edge, providing a directional flexibility in the horizontal plane.

The relative heights of the anterior and posterior arches of the anterior and posterior segments 10 and 40, respectively, are fixed and represent 12% to 25% of the intercoinmissural diameter 100 of the ring, for example an amplitude of 2,5 mm to 7 mm in the original resting shape of the unstressed prosthetic ring. In order better to conform to the anatomy of the native mitral annulus, the height of the anterior arch is preferably greater than the height of the posterior arch, with the difference ranging from 1 to 4 mm.

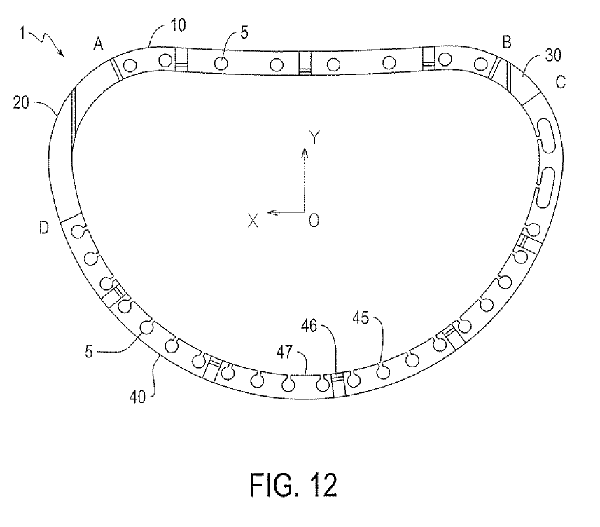

In embodiments, the upwardly arched anterior segment 10 may be provided with slits 12 that are disposed on the superior face of the anterior segment 10, close to its ends A and B, providing directional flexibility in the frontal plane, as shown in FIGS. 8-13B. When a downward force in the Z direction is applied to the ring 1, the slits 12 open, rendering the anterior segment 10 flexible in this direction, as shown in FIGS. 13A and 13B. The lateral parts of the anterior segment and consequently the lateral segments 20 and 30, at least in the portions of the lateral segments 20 and 30 that are close to the ends of the anterior segment 10, move downward and inward, increasing the height of the anterior segment 10 and concomitantly reducing the intercommissural distance 100 of the ring. This represents one of the physiological deformations of the native mitral annulus during the cardiac cycle. When the downwardly directed forces disappear, the prosthetic ring 1 passively returns to its initial position and resumes its resting shape because of its elastic properties.

When an upward force in the Z direction is applied to the lateral parts of the anterior segment 10, the slits 12 close and bring the sub-segments 13 into contact with each other, which thereby stiffens the body of the anterior segment 10 and prevents its upward movement. This rigidity resists any further shortening of the height of the anterior segment and thus also prevents any segmentary flattening of the native mitral annulus.

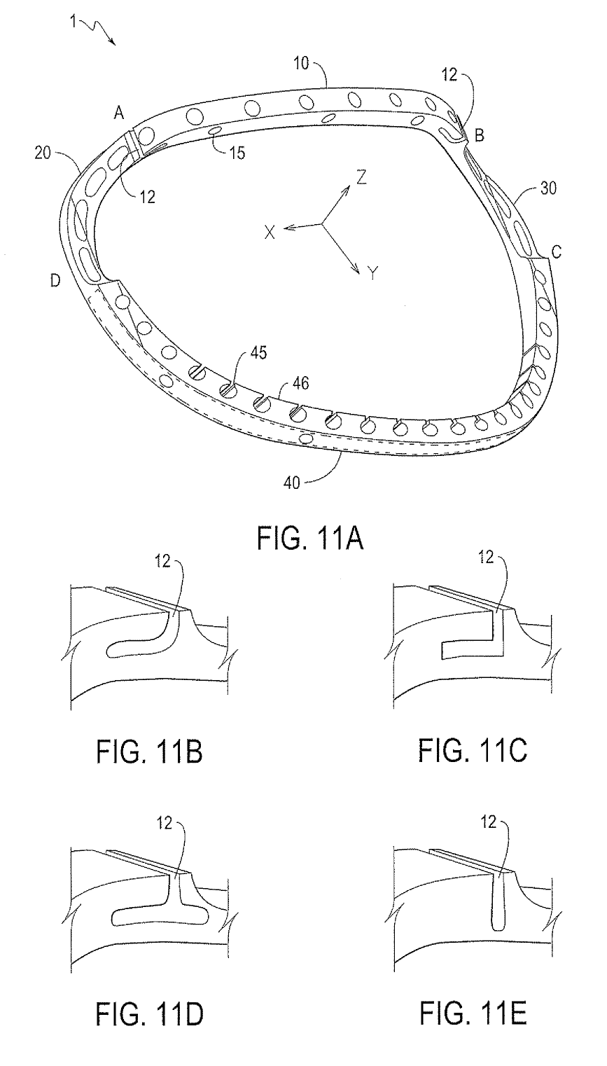

The above description discusses embodiments that include multiple slits 12 disposed on the superior face of the anterior segment 10. However, in some embodiments, only a single slit 12 is disposed on the superior face of the anterior segment 10. In embodiments with multiple slits 12, the slits 12 may have equal or unequal depths, as shown in FIGS. 11A-11E. The slits 12 can be formed inside the body through the anterior face in a straight manner, or slightly curved or angled in the core of the body and directed towards one or directed toward the same or different directions along the major axis of the anterior segment 10, as shown in FIGS. 11A-11E.

The original resting shape of the prosthetic mitral ring 1 preferably corresponds to the morphology of the native mitral annulus in diastole, namely in its more flattened form during the cardiac cycle.

It is important to understand that, while the heights of the anterior and posterior arcuate segments 10 and 40 are fixed, the height of the prosthetic ring 1 varies during the cardiac cycle according to the lower displacement of the lateral segments 20 and 30 that are subjected to systolic forces applied to them during the cardiac cycle. The great flexibility of these lateral segments 20 and 30 enables the prosthetic ring 1 to adapt passively to the height variations of the native mitral annulus.

The height of the prosthetic ring 1 in systole is the sum of the height of the arcuate segment in question and the displacement of the lowest point of the lateral segment. Thus, the height of the prosthetic ring 1 in systole becomes greater than the height of the anterior and posterior arcuate segments 10 and 40, and increases for example, from 4 to 6 mm depending on the forces applied to the lateral segments 20 and 30. Modeling studies of a prosthetic ring 1 have demonstrated these characteristics when the prosthetic ring is subjected to physiological forces that are applied to the lateral segments 20 and 30.

The relative heights of the prosthetic ring in systole preferably represent 25% to 40% of the length of the intercommissural diameter 100 of the prosthetic ring.

Because of the pliable nature of the lateral segments 20 and 30, the prosthetic ring 1, which is symmetrical in its resting state, may become asymmetrical when forces applied to the lateral segments 20 and 30 are unequal.

In embodiments, the lateral segments 20 and 30 may have unequal lengths. For example, the distance between A and D may be shorter than the distance between B and C, or vice versa, rendering the ring structurally asymmetrical. This allows the surgeon to adapt to situations where for example there is a lack or loss of leaflet tissue, as encountered in endocarditis or other pathological presentation.

In embodiments, the prosthetic ring 1 may be continuous. In such configurations, when forces that are typical during systole act on the right lateral segment 30, the right lateral segment 30 may be displaced in a downward manner due to its highly supple and flexible nature, as shown in FIG. 8.

In embodiments, the prosthetic ring 1 may be discontinuous, preferably at junction B. In such configurations, when forces that are typical during systole act on the right lateral segment 30, the right lateral segment 30 may be displaced in a downward manner due to its highly supple and flexible nature, as shown in FIGS. 4 and 6.

In embodiments, the prosthetic ring may be discontinuous at junctions A and/or B. In this configuration, when subjected to forces during systole, the right lateral segment 30 and/or the left lateral segment 20, may be displaced in a downward direction due to their highly supple and flexible nature, as shown in FIG. 7.

In embodiments, a plurality of holes 5 may be formed through the lower and upper faces all around of the ring body, or particularly in the posterior and/or anterior segments. The holes 5, which are generally but not necessarily evenly distributed, enable the sutures, staples or other links for securing the prosthetic ring 1 to the native mitral annulus to pass through. The holes 5 are preferably numerous and closely spaced, so that it is not necessary to flag them by marks on a sleeve that may envelop the body. These holes 5 allow excellent anchoring of the prosthetic ring 1 to the native mitral annulus and eliminate the need for an additional silicone layer, which is generally present in conventional rings between the outer envelope and the body of the ring. The arrangement of holes 5, along with the small dimensions in cross section of the prosthetic ring body, contributes to the very low profile of the ring 1. For example, embodiments of the current invention provide a prosthetic ring that is suitable for implantation in children, and capable of reducing the fibrosis reaction around the ring after implantation.

In embodiments in which the body of the ring 1 has securing holes 5, the latter may be opened by the slits 15 or 45 disposed on the inside face of the ring body. Depending on their location on the ring body, the holes 5 may receive the slits or not receive them. The securing holes may have different geometric shapes, have different dimensions, and be arranged in various configurations.

The body 8 of the prosthetic ring 1 may be made of a biocompatible material, for example, of the polyethylene or polyurethane type. In embodiments, the anterior and posterior segments 10 and 40 are formed with a metal reinforcement strip 6, that is made of nitinol, or another elastic metal or alloy, included in the biocompatible plastic material of the body 8 in order to stiffen the latter in the frontal plane and maintain the height of the arch of the segments, as shown in FIGS. 7A-7E. The reinforcement strips 6 may be very thin in comparison to their height, whereby they do not alter the mechanical characteristics in the horizontal plane of the anterior and posterior segments 10 and 40.

In embodiments, the prosthetic ring 1 is covered with a flexible DACRON® polyester sheath (not shown), which may be used as an additional or alternative element for securing the prosthetic ring 1 to the native mitral annulus.

In embodiments, the body 8 of the prosthetic ring 1 may or may not have securing holes 5, but is surrounded by a layer of silicone 9 and preferably also a DACRON® polyester sleeve in order to enable it to be secured to the native mitral annulus. FIG. 7A is schematic representation of an example prosthetic ring 1, depicted with four cross-sectional planes 7B-7E. FIG. 7B is an example cross section of lateral segments 30 that depicts a structural configuration of layer of silicone 9 surrounding the body 8, which corresponds to the cross-sectional plane 7B that is taken in a hole 5 of the lateral segment 30. FIG. 7C is an examples cross section of the posterior segment 40 that depicts structural configurations of the strip 6 embedded in the body 8. In particular, FIG. 7C corresponds to the cross-sectional plane 7C that depicts the structure of posterior segment 40 near the arch of the posterior segment 40 and taken in a slit 45 and hole 5. A strip 6 is embedded in the body 8 and a layer of silicone surrounds the slit 45, hole 5, and the body 8. The configuration depicted in FIG. 7D is an example cross section of the posterior segment 40 near the junction D. In particular, FIG. 7D corresponds to the cross-sectional plane 7D, and depicts a layer of silicone 9 surrounding the body 8, which includes a strip 6 embedded therein. FIG. 7E is an example cross section of the anterior segment 10 that depicts a structural configuration across the cross-sectional plane 7E. A strip 6 is embedded in the body 8, and the body 8 is surrounded by a layer of silicone 9.

In embodiments, the body of the prosthetic ring 1 is made of the same flexible biocompatible material such as nitinol, or other elastic metallic alloy, in its four segments. In such a configuration, the lateral segments 20 and 30 are very thin in order to offer the smallest resistance to their deformation.

In embodiments, the anterior and posterior segments 10 and 40 are made of a hard, non-deformable material such as porous titanium, and are extended by flexible pliable lateral segments 20 and 30 that are made of biocompatible plastic, preferably with a DACRON® polyester type sheath enveloping all of the segments.

In embodiments, anterior and posterior porous titanium segments 10 and 40 are perforated with holes 5 for securing them to the native mitral annulus and are not covered with a DACRON® sheath. The porous titanium can be repopulated with endocardial cells and may include tissue compatibility characteristics that generate a minimal inflammatory reaction that is generally confined to a scarring reaction. The properties of porous titanium make it a material of choice for a prosthetic mitral ring.

In embodiments, the lateral segments 20 and 30 may be formed solely by the sheath, whether or not the sheath ensheathes the anterior and posterior segments 10 and 40. In such a configuration, the sheath performs the functions of the lateral segments 20 and 30, while providing a securing function.

In embodiments, the lateral segments 20 and 30 are formed by an inextensible, pliable, highly flexible material made of a biocompatible molded, woven, or knitted material (e.g., polyethylene or polyurethane).

In embodiments, the lateral segments 20 and 30 are in the form of thin, rigid or semi-rigid, juxtaposed elements that are disposed transversely to the major axis of the ring 1, separated by empty space or not so separated and joined together by the anterior and posterior segments 10 and 40, respectively, in the manner of a Roman blind (i.e., the lateral segments 20 and 30 form right angles with and are foldable towards the anterior segment 10 and posterior segment).

The lateral flexible and pliable segments 20 and 30 respectively have a length representing about 10% to 30% of the length of the septolateral diameter 110 of the ring when the intercommissural diameter 100 to septolateral diameter 110 ratio is 3.2:4 (80%). When the intercommissural diameter 100 to septolateral diameter 110 ratio is greater than 3.2:4, the increase in length of the septolateral diameter 110 of the ring is essentially at the expense of the lateral pliable segments 20 and 30, the length of the lateral flexible and pliable segments 20 and 30 may represent about 30% to 60% of the length of the septolateral diameter 110, and the prosthetic ring 1 retains the general appearance of a capital letter “D.”

The apparatus and methods described above with reference to the various embodiments are merely examples. It goes without saying that they are not confined to the depicted embodiments. While various features have been described in conjunction with the examples outlined above, various alternatives, modifications, variations, and/or improvements of those features and/or examples may be possible. Accordingly, the examples, as set forth above, are intended to be illustrative. Various changes may be made without departing from the broad spirit and scope of the underlying principles.

What is claimed is:

1. A three-dimensional mitral prosthetic ring having a substantially hyperbolic and parabolic shape, in a closed position, the prosthetic ring substantially forming a saddle shape, the prosthetic mitral ring having a ring body comprising:
an anterior segment, most of the anterior segment being rigid along a frontal plane and having slits arranged in an upper face of the anterior segment on lateral parts of the anterior segment that provide selective directional flexibility and rigidity properties in the frontal plane, and semi-rigid or rigid along a horizontal plane, arched upward along the frontal plane, securable between trigones on an anterior part of a native mitral annulus;
a left lateral segment and a right lateral segment, the lateral segments being flat or slightly curved, supple, and pliable, such that the mitral prosthetic ring has a systolic height greater than a height of the anterior arch, and being implantable in commissural zones and in adjacent zones that are adjacent to commissures of a native mitral valve; and
a posterior segment, the posterior segment being upwardly arched along the frontal plane, a height of the arch of the posterior segment being less than or equal to the height of the arch of the anterior segment, and having slits disposed in an inside edge of the posterior segment that provide selective directional flexibility and rigidity properties in the horizontal plane, the posterior segment being securable to the posterior part of a native mitral annulus.

2. The prosthetic mitral ring according to claim 1, wherein the ring includes securing holes that pass through upper and lower faces of the body of the ring, such that the body of the ring is securable directly to cardiac tissue.

3. The prosthetic mitral ring according to claim 2, wherein the body of the ring is securable to cardiac tissue by sutures.

4. The prosthetic mitral ring according to claim 2, wherein the slits are distributed over part of the posterior segment, and have unequal depths that open some but not all of the securing holes that pass through the posterior segment.

5. The prosthetic mitral ring according to claim 2, wherein the slits are distributed over part of the posterior segment, and have equal depths that open some but not all of the securing holes that pass through the posterior segment.

6. The prosthetic mitral ring according to claim 2, further comprising slits on an inside face of the anterior segment that provide selective directional flexibility and rigidity to the anterior segment in the horizontal plane, said slits being distributed over part of the anterior segment, and having unequal depths that open some but not all of the securing holes that pass through the anterior segment.

7. The prosthetic mitral ring according to claim 2, farther comprising slits on an inside face of the anterior segment that provide selective directional flexibility and rigidity to the anterior segment in the horizontal plane, said slits being distributed over part of the anterior segment, and having equal depths that open some but not all of the securing holes that pass through the anterior segment.

8. The prosthetic mitral ring according to claim 1, further comprising slits on the upper face of the anterior segment that provide selective directional flexibility and rigidity to the anterior segment in the frontal plane, said slits being distributed over a part of the anterior segment, and being straight, curved, or angled, having uneven depths or lengths, such that the anterior height of the prosthetic mitral ring may increase by 0 to 6 mm relative to the height of the anterior arch, symmetrically or asymmetrically, during a cardiac cycle of a native mitral annulus to which the ring may be attached.

9. The prosthetic mitral ring according to claim 1, further comprising slits on the upper face of the anterior segment and that provide selective directional flexibility and rigidity to the anterior segment in the frontal plane, said slits being distributed over a part of the anterior segment, and being straight, curved, or angled, having even depths or lengths, such that the anterior height of the prosthetic mitral ring may increase by 0 to 6 mm relative to the height of the anterior arch, symmetrically or asymmetrically, during a cardiac cycle of a native mitral annulus to which the ring may be attached

10. The prosthetic mitral ring according to claim 1, the lateral segments being flexible and non-extendable, such that the anterior height of the prosthetic mitral ring is increasable by 0 to 6 mm relative to the height of the anterior arch, symmetrically or asymmetrically, during a cardiac cycle of a native mitral annulus to which the ring may be attached.

11. The prosthetic mitral ring according to claim 1, comprising a first junction between a first end of the anterior segment and a first end of the left lateral segment, a second junction between a second end of the anterior segment and a first end of the right lateral segment, a third junction between a first end of the posterior segment and a second end of the left lateral segment, and a fourth junction between a second end of the posterior segment and a second end of the right lateral segment.

12. The prosthetic mitral ring according to claim 1, comprising a first junction between a first end of the anterior segment and a first end of the left lateral segment, a second junction between a first end of the posterior segment and a second end of the left lateral segment, and a third junction between a second end of the posterior segment and a first end of the right lateral segment, but no junction between a second end of the anterior segment and a second end of the right lateral segment, such that mobility of the right lateral segment is increased in a right anterior commissural zone by relative passive offset of the anterior and right lateral segments.

13. The prosthetic mitral ring according to claim 1, comprising a first junction between a first end of the posterior segment and an end of the left lateral segment, and a second junction between a second end of the posterior segment and an end of the right lateral segment, but no junctions between the anterior segment and the lateral segments, such that the mobility of the lateral segments is increased in right anterior and left anterior commissural zones by relative passive offset of the lateral segments that are adjacent to the anterior segment.

14. The prosthetic mitral ring according to claim 1, wherein the lateral segments have unequal lengths, such that the ring is asymmetric.

15. The prosthetic mitral ring according to claim 1, wherein the lateral segments have equal lengths, such that the ring is symetric.

16. The prosthetic mitral ring according to claim 1, wherein the lengths of the lateral segments are in the range of 10% to 60% of the length of a septolateral diameter of the ring.

17. The prosthetic mitral ring according to claim 1, wherein the saddle shape and a size of the ring are configured based on a ratio between a septolateral diameter and an intercommissural diameter of the ring.

18. The prosthetic mitral ring according to claim 17 a ratio between the septolateral diameter and the intercommissural diameter is in the range of 0.85:1 to 1:1.

19. The prosthetic mitral ring according to claim 17, wherein a ratio between the septolateral diameter and the intercommissural diameter is 1:1.

20. The prosthetic mitral ring according to claim 17, wherein a ratio between the septolateral diameter and the intercommissural diameter is in the range of 1:1 to 1.15:1.

21. The prosthetic mitral ring according to claim 1, wherein the anterior and posterior segments are provided with metal reinforcements to allow stiffening of the ring body along the frontal plane and to maintain the height of the arches.

22. The prosthetic mitral ring according to claim 10, wherein the metal reinforcements are formed of a biocompatible elastic metal or alloy.

23. The prosthetic mitral ring according to claim 10, wherein the metal reinforcements are formed of nitinol.

24. The prosthetic mitral ring according to claim 1, wherein the segments are made of a single biocompatible material.

25. The prosthetic mitral ring according to claim 1, the segments being covered with a peripheral securing sheath made of polyester.

26. The prosthetic mitral ring according to claim 1, wherein the anterior and posterior segments are made of porous titanium or porous nitinol and are formed with said securing holes, and the lateral segments are made of a supple, pliable biocompatible material.

27. The prosthetic mitral ring according to claim 14, wherein the biocompatible material is selected from the group consisting of polyethylene and polyurethane.

28. The prosthetic mitral ring according to claim 1, having no securing holes passing through the upper and lower faces of the ring body, and having a peripheral sheath surrounding the ring body over all or part of the length of the ring body such that the ring body may be sutured directly to cardiac tissue.

29. The prosthetic mitral ring according to claim 1, wherein the ring body has a very low profile such that the anterior and posterior segments have a width that is less than or equal to 2 mm and a thickness that is less than or equal to 1.5 mm, and such that the lateral segments have a thickness that is less than or equal to 0.2 mm and a width that is less than or equal to 2 mm.

30. A method for performing mitral annuloplasty, comprising determining a size for the prosthetic mitral ring of claim 1 to be implanted in a patient, wherein the size of the prosthetic ring is determined during diastolic superimposition of the native mitral annulus of the patient and after injecting a normal saline solution into a left ventricle of the patient.

31. A method according to claim 30, wherein the size is determined such that lengths of the lateral segments are in the range of 10% to 60% of a septolateral diameter of the ring.

32. A method according to claim 30, wherein the size is determined in accordance with a ratio between a calculated septolateral diameter and a measured intercommissural diameter of a native mitral annulus of the patient.

33. A method according to claim 30, wherein the size is determined such that a ratio between a septolateral diameter and an intercommissural diameter of the ring is less than 1:1.

34. A method according to claim 30, wherein the size is determined such that a ratio between a septolateral diameter and an intercommissural diameter of the ring is 1:1.

35. A method according to claim 30, wherein the size is determined such that a ratio between a septolateral diameter and an intercommissural diameter of the ring is greater than 1: 1.

36. A method according to claim 30, wherein the size is determined such that a ratio between a calculated septolateral diameter and a measured intercommissural diameter of a native mitral annulus of the patient is 1:1, and 80% to 90% of the sum of maximum heights of anterior and posterior leaflets is equal to the length of the intercommissural diameter.

37. A method according to claim 30, wherein the size is determined such that a ratio between a calculated septolateral diameter and a measured intercommissural diameter of a native mitral annulus of the patient is less than 1:1, and 80% to 90% of the sum of maximum heights of anterior and posterior leaflets is less than the length of the intercommissural diameter.

38. A method according claim 30, wherein the size is determined such that a ratio between a calculated septolateral diameter and a measured intercommissural diameter of a native mitral annulus of the patient is greater than 1:1, and 80% to 90% of the sum of maximum heights of anterior and posterior leaflets is greater than the length of the intercommissural diameter.

39. A method for the treatment of a native mitral annulus, comprising:
measuring an intercommissural diameter of the mitral annulus;
measuring heights of anterior and posterior leaflets of the mitral annulus;
calculating a septolateral diameter of the mitral annulus based on the measured heights of the anterior and posterior leaflets; and
securing a prosthetic mitral ring to the mitral annulus, wherein the mitral ring has an intercommissural diameter that corresponds to the measured intercommissural diameter of the mitral annulus and has a septolateral diameter that corresponds to the calculated septolateral diameter of the mitral annulus.

40. The method of claim 34, wherein the calculated septolateral diameter of the mitral annulus is calculated to be 80-90% of the sum of the measured heights of the anterior and posterior leaflets.
